(19) United States
(12) Patent Application Publication
(10) Pub. No.: US 2022/0280227 A1
(43) Pub. Date: Sep. 8, 2022

(54) END EFFECTOR DRIVE MECHANISMS FOR SURGICAL INSTRUMENTS SUCH AS FOR USE IN ROBOTIC SURGICAL SYSTEMS

(71) Applicant: Covidien LP, Mansfield, MA (US)

(72) Inventor: Zachary S. Heiliger, Nederland, CO (US)

(21) Appl. No.: 17/190,072

(22) Filed: Mar. 2, 2021

(51) Int. Cl. A61B 18/14; A61B 34/30

(52) U.S. Cl. CPC A61B 18/1447; A61B 34/30; A61B 2017/2936

(57) ABSTRACT

A surgical instrument includes first and second jaw members each having a proximal portion including a flag defining a cam groove having a hemispherical cross-sectional, and a distal portion defining a tissue-contacting surface. A cam drive mechanism includes a drive rod and a drive ball disposed at a distal end portion of the drive rod. The drive ball is captured within the cam grooves between the flags such that translation of the drive rod in a first direction slides the cam ball through the cam grooves to pivot the distal portion of at least one of the first or second jaw members relative to the distal portion of the other of the first or second jaw members towards an approximated position for grasping tissue between the tissue-contacting surfaces of the distal portions of the first and second jaw members.

### FIELD

The present disclosure relates to surgical instruments and, more specifically, to end effector drive mechanisms for surgical instruments such as for use in robotic surgical systems.

### BACKGROUND

Robotic surgical systems are increasingly utilized in various different surgical procedures. Some robotic surgical systems include a console supporting a robotic arm. One or more different surgical instruments may be configured for use with the robotic surgical system and selectively mountable to the robotic arm. The robotic arm provides one or more inputs to the mounted surgical instrument to enable operation of the mounted surgical instrument.

A surgical forceps, one type of instrument capable of being utilized with a robotic surgical system, relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is treated, the tissue is severed using a cutting element. Accordingly, electrosurgical forceps are designed to incorporate a cutting element to effectively sever treated tissue. Alternatively, energy-based, e.g., thermal, electrical, ultrasonic, etc., cutting mechanisms may be implemented.

### SUMMARY

As used herein, the term “distal” refers to the portion that is being described which is farther from an operator (whether a human surgeon or a surgical robot), while the term “proximal” refers to the portion that is being described which is closer to the operator. The terms “about,” substantially,” and the like, as utilized herein, are meant to account for manufacturing, material, environmental, use, and/or measurement tolerances and variations, and in any event may encompass differences of up to 10%. Further, to the extent consistent, any of the aspects described herein may be used in conjunction with any or all of the other aspects described herein.

Provided in accordance with aspects of the present disclosure is a surgical instrument including a first jaw member, a second jaw member, and a cam drive mechanism. The first jaw member defines a proximal portion including at least a first flag and a distal portion defining a tissue-contacting surface. The first flag defines a first cam groove having a hemispherical cross-sectional configuration. The second jaw member defines a proximal portion including at least a second flag and a distal portion defining a tissue-contacting surface. The second flag defines a second cam groove having a hemispherical cross-sectional configuration. The first and second jaw members are pivotably coupled to one another. The cam drive mechanism includes a drive rod and a drive ball disposed at a distal end portion of the drive rod. The drive ball is captured within the first and second cam grooves between the first and second flags. Translation of the rive rod in a first direction slides the cam ball through the cam grooves to pivot the distal portion of at least one of the first or second jaw members relative to the distal portion of the other of the first or second jaw members towards an approximated position for grasping tissue between the tissue-contacting surfaces of the distal portions of the first and second jaw members.

In an aspect of the present disclosure, the surgical instrument further includes a shaft having a distal segment. In such aspects, the proximal portion of the second jaw member may be fixed to the distal segment of the shaft. Alternatively or additionally, the shaft may further include an articulating section proximal to the distal segment.

In another aspect of the present disclosure, the proximal portion of the second jaw member further includes a third flag spaced-apart from the second flag. The first flag may be disposed between the second and third flags.

In still another aspect of the present disclosure, the first flag substantially occupies a space defined between the second and third flags to maintain the drive ball captured within the first and second cam grooves between the first and second flags.

In yet another aspect of the present disclosure, the first and second cam grooves at least partially intersect to form a spherical cavity for receipt of the drive ball therein.

In still yet another aspect of the present disclosure, at least one of the tissue-contacting surfaces is adapted to connect to a source of electrosurgical energy for treating tissue grasped between the tissue-contacting surfaces.

Another surgical instrument provided in accordance with aspects of the present disclosure includes a housing, a shaft extending distally from the housing, an end effector assembly disposed at a distal end of the shaft and including first and second jaw members, and a cam drive mechanism. The first and second jaw members and the cam drive mechanism may be configured according to any of the aspects detailed above or otherwise herein.

In an aspect of the present disclosure, the housing includes a jaw drive mechanism disposed therein that is operably coupled to the drive rod.

In another aspect of the present disclosure, the housing is configured to mount on a surgical robot configured to operate the jaw drive mechanism to translate the drive rod.

### BRIEF DESCRIPTION OF THE DRAWINGS

Various aspects and features of the present disclosure are described hereinbelow with reference to the drawings wherein:

FIG. 1 is a perspective view of a surgical instrument in accordance with the present disclosure configured for mounting on a robotic arm of a robotic surgical system;

FIG. 2 is a rear perspective view of a proximal portion of the surgical instrument of FIG. 1 with an outer housing removed;

FIG. 3 is a schematic illustration of an exemplary robotic surgical system configured to releasably receive the surgical instrument of FIG. 1;

FIG. 4A is an enlarged, side view of an end effector assembly configured for use with the surgical instrument of FIG. 1 or any other suitable surgical instrument;

FIG. 4B is a transverse, cross-sectional view of a proximal portion of the end effector assembly of FIG. 4A;

FIG. 5 is a perspective view of a jaw frame of one of the jaw members of the end effector assembly of FIG. 4A;

FIG. 6A is a perspective view of the other jaw member of the end effector assembly of FIG. 4A;

FIG. 6B is a side view of the jaw member of FIG. 6A including a distal portion of a drive mechanism operably coupled thereto;

FIG. 7A is a top view of the end effector assembly of FIG. 4A including the distal portion of the drive mechanism coupled thereto with the jaw members disposed in a spaced-apart position; and

FIG. 7B is a top view of the end effector assembly of FIG. 4A including the distal portion of the drive mechanism coupled thereto with the jaw members disposed in an approximated.

### DETAILED DESCRIPTION

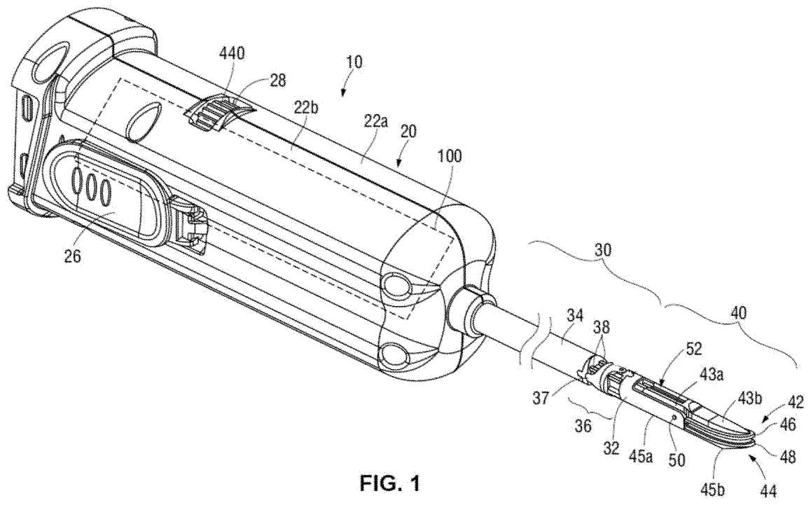

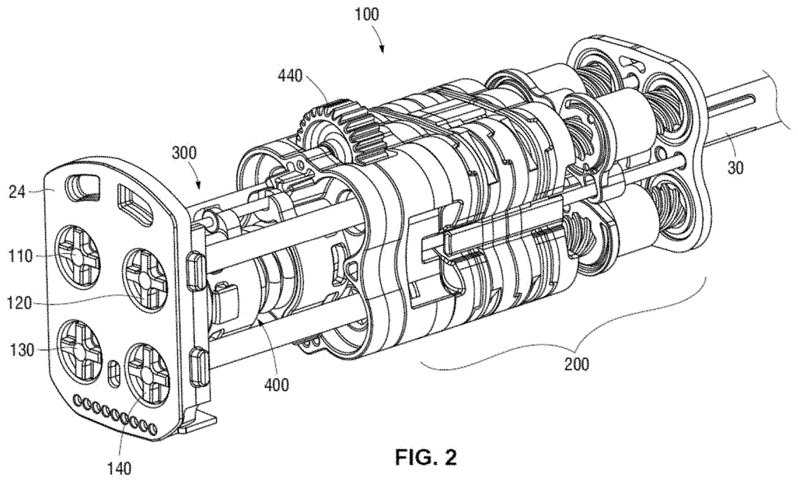

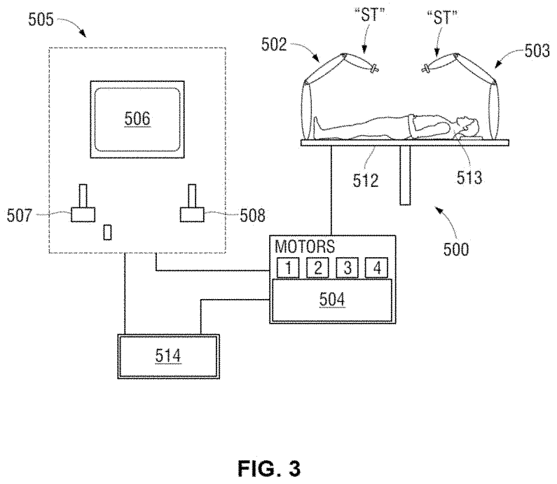

Referring to FIGS. 1 and 2, a surgical instrument 10 provided in accordance with the present disclosure generally includes a housing 20, a shaft 30 extending distally from housing 20, an end effector assembly 40 extending distally from shaft 30, and an actuation assembly 100 disposed within housing 20 and operably associated with shaft 30 and end effector assembly 40. Instrument 10 is detailed herein as an articulating electrosurgical forceps configured for use with a robotic surgical system, e.g., robotic surgical system 500 (FIG. 3). However, the aspects and features of instrument 10 provided in accordance with the present disclosure, detailed below, are equally applicable for use with other suitable surgical instruments (including non-robotic surgical instrument) and/or in other suitable surgical systems (including non-robotic surgical systems).

Housing 20 of instrument 10 includes first and second body portion 22a, 22b and a proximal face plate 24 (FIG. 2) that cooperate to enclose actuation assembly 100 therein. Proximal face plate 24 includes apertures defined therein through which inputs 110-140 of actuation assembly 100 extend. A pair of latch levers 26 (only one of which is illustrated in FIG. 1) extend outwardly from opposing sides of housing 20 and enables releasable engagement (directly or indirectly) of housing 20 with a robotic arm of a surgical system, e.g., robotic surgical system 500 (FIG. 3). An aperture 28 defined through housing 20 permits thumbwheel 440 to extend therethrough to enable manual manipulation of thumbwheel 440 from the exterior of housing 20 to permit manual opening and closing of end effector assembly 40.

Shaft 30 of instrument 10 includes a distal segment 32, a proximal segment 34, and an articulating section 36 disposed between the distal and proximal segments 32, 34, respectively. Articulating section 36 includes one or more articulating components 37, e.g., links, joints, etc. A plurality of articulation cables 38, e.g., four (4) articulation cables, or other suitable actuators, extends through articulating section 36. More specifically, articulation cables 38 are operably coupled to distal segment 32 of shaft 30 at the distal ends thereof and extend proximally from distal segment 32 of shaft 30, through articulating section 36 of shaft 30 and proximal segment 34 of shaft 30, and into housing 20, wherein articulation cables 38 operably couple with an articulation assembly 200 of actuation assembly 100 to enable selective articulation of distal segment 32 (and, thus end effector assembly 40) relative to proximal segment 34 and housing 20, e.g., about at least two axes of articulation (yaw and pitch articulation, for example). Articulation cables 38 are arranged in a generally rectangular configuration, although other suitable configurations are also contemplated.

With respect to articulation of end effector assembly 40 relative to proximal segment 34 of shaft 30, actuation of articulation cables 38 is effected in pairs. More specifically, in order to pitch end effector assembly 40, the upper pair of cables 38 is actuated in a similar manner while the lower pair of cables 38 is actuated in a similar manner relative to one another but an opposite manner relative to the upper pair of cables 38. With respect to yaw articulation, the right pair of cables 38 is actuated in a similar manner while the left pair of cables 38 is actuated in a similar manner relative to one another but an opposite manner relative to the right pair of cables 38.

Distal segment 32 of shaft 30 defines a clevis portion of end effector assembly 40 that supports first and second jaw members 42, 44, respectively. Each jaw member 42, 44 includes a proximal extension portion 43a, 45a and a distal body portion 43b, 45b, respectively. Distal body portions 43b, 45b define opposed tissue-contacting surfaces 46, 48, respectively. Proximal extension portions 43a, 45a are pivotably coupled to one another about a pivot pin 50 and are operably coupled to one another via a cam drive mechanism 52 (described in greater detail below) to enable pivoting of jaw member 42 relative to jaw member 44 and distal segment 32 of shaft 30 between a spaced-apart position (e.g., an open position of end effector assembly 40) and an approximated position (e.g., a closed position of end effector assembly 40) for grasping tissue between tissue-contacting surfaces 46, 48. As an alternative to this unilateral configuration, a bilateral configuration may be provided whereby both jaw members 42, 44 are pivotable relative to one another and distal segment 32 of shaft 30.

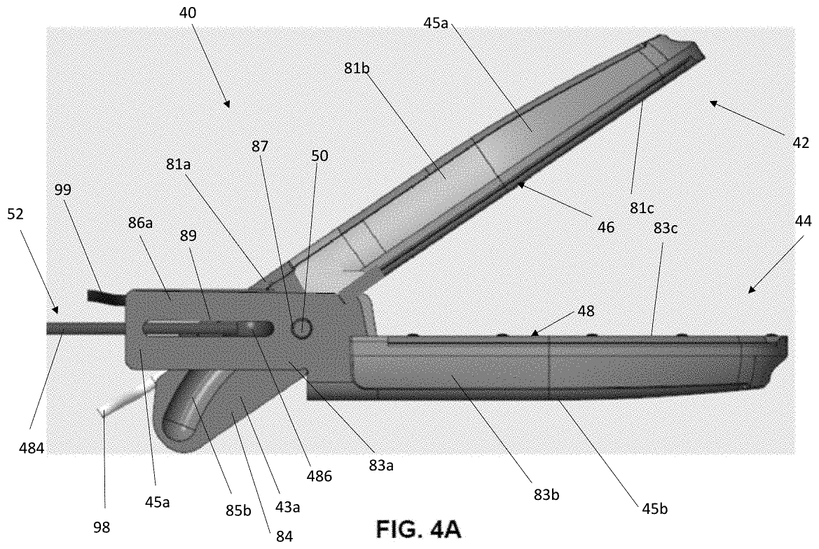

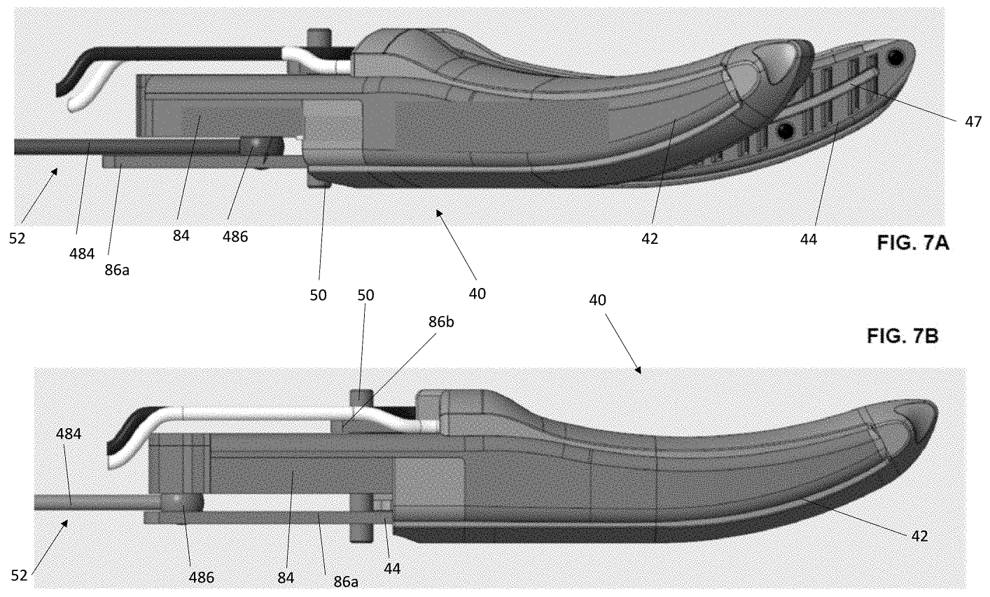

A longitudinally-extending channel 47 (FIG. 7A) of jaw member 44 and/or a corresponding channel (not shown) of jaw member 42, are defined through tissue-contacting surfaces 46, 48, respectively, of jaw members 42, 44. A translating cutting element 72 (FIG. 4B) is provided and selectively advanceable to enable cutting of tissue grasped between tissue-contacting surfaces 46, 48 of jaw members 42, 44, respectively. A cutting drive assembly 300 of actuation assembly 100 provides for selective actuation of cutting element 72 (FIG. 4B) to translate cutting element 72 (FIG. 4B) through channel(s) 47 (FIG. 7A) of jaw members 42, 44 to cut tissue grasped between tissue-contacting surfaces 46, 48. Cutting drive assembly 300 is operably coupled to third input 130 of actuation assembly 100 such that, upon receipt of appropriate rotational input into third input 130, cutting drive assembly 300 advances the cutting element 72 (FIG. 4B) between jaw members 42, 44 to cut tissue grasped between tissue-contacting surfaces 46, 48.

Continuing with reference to FIGS. 1 and 2, a drive rod 484 (FIGS. 7A and 7B) of cam drive mechanism 52 is operably coupled to end effector assembly 40 such that longitudinal actuation of drive rod 484 (FIGS. 7A and 7B) pivots jaw member 42 relative to jaw member 44 between the spaced-apart and approximated positions, as detailed below. More specifically, urging drive rod 484 (FIGS. 7A and 7B) proximally pivots jaw member 42 relative to jaw member 44 towards the approximated position while urging drive rod 484 (FIGS. 7A and 7B) distally pivots jaw member 42 relative to jaw member 44 towards the spaced-apart position. However, the reverse configuration is also contemplated. Drive rod 484 (FIGS. 7A and 7B) extends proximally from end effector assembly 40 through shaft 30 and into housing 20 wherein drive rod 484 (FIGS. 7A and 7B) is operably coupled with a jaw drive assembly 400 of actuation assembly 100 to enable selective actuation of end effector assembly 40 to grasp tissue therebetween and apply a closure force within an appropriate jaw closure force range, e.g., in response to an appropriate rotational input into fourth input 140.

Tissue-contacting surfaces 46, 48 of jaw members 42, 44, respectively, are at least partially formed from an electrically conductive material and are energizable to different potentials to enable the conduction of electrical energy through tissue grasped therebetween, although tissue-contacting surfaces 46, 48 may alternatively be configured to supply any suitable energy, e.g., thermal, microwave, light, ultrasonic, etc., through tissue grasped therebetween for energy-based tissue treatment. Instrument 10 defines conductive pathways extending through housing 20 and shaft 30 to end effector assembly 40 that may include lead wires, contacts, and/or electrically-conductive components to enable electrical connection of tissue-contacting surfaces 46, 48 of jaw members 42, 44, respectively, to an energy source (not shown), e.g., an electrosurgical generator via an electrosurgical cable extending therebetween, for supplying energy to tissue-contacting surfaces 46, 48 to treat, e.g., seal, tissue grasped between tissue-contacting surfaces 46, 48. The electrically conductive pathways to tissue-contacting surfaces 46, 48 of jaw members 42, 44, are illustrated, for example, as respective first and second lead wires 98, 99 (see FIG. 4A).

Actuation assembly 100 is disposed within housing 20 and includes articulation assembly 200, cutting drive assembly 300, and jaw drive assembly 400. Articulation assembly 200 is operably coupled between first and second inputs 110, 120, respectively, of actuation assembly 100 and articulation cables 38 such that, upon receipt of appropriate rotational inputs into first and/or second inputs 110, 120, articulation assembly 200 manipulates cables 38 (FIG. 1) to articulate end effector assembly 40 in a desired direction, e.g., to pitch and/or yaw end effector assembly 40. Cutting drive assembly 300, as noted above, enables reciprocation of the cutting element 72 (FIG. 4B) between jaw members 42, 44 to cut tissue grasped between tissue-contacting surfaces 46, 48 in response to receipt of appropriate rotational input into third input 130. Jaw drive assembly 400 is operably coupled between fourth input 140 of actuation assembly 100 and drive rod 484 (FIGS. 7A and 7B) such that, upon receipt of appropriate rotational input into fourth input 140, jaw drive assembly 400 pivots jaw members 42, 44 between the spaced-apart and approximated positions to grasp tissue therebetween and apply a closure force within an appropriate closure force range.

Actuation assembly 100 is configured to operably interface with a robotic surgical system 500 (FIG. 3) when instrument 10 is mounted on robotic surgical system 500 (FIG. 3), to enable robotic operation of actuation assembly 100 to provide the above-detailed functionality. That is, robotic surgical system 500 (FIG. 3) selectively provides rotational inputs to inputs 110-140 of actuation assembly 100 to articulate end effector assembly 40, grasp tissue between jaw members 42, 44, and/or cut tissue grasped between jaw members 42, 44. However, it is also contemplated that actuation assembly 100 be configured to interface with any other suitable surgical system, e.g., a manual surgical handle, a powered surgical handle, etc. For the purposes herein, robotic surgical system 500 (FIG. 3) is generally described.

Turning to FIG. 3, robotic surgical system 500 is configured for use in accordance with the present disclosure. Aspects and features of robotic surgical system 500 not germane to the understanding of the present disclosure are omitted to avoid obscuring the aspects and features of the present disclosure in unnecessary detail.

Robotic surgical system 500 generally includes a plurality of robot arms 502, 503; a control device 504; and an operating console 505 coupled with control device 504. Operating console 505 may include a display device 506, which may be set up in particular to display three-dimensional images; and manual input devices 507, 508, by means of which a person, e.g., a surgeon, may be able to telemanipulate robot arms 502, 503 in a first operating mode. Robotic surgical system 500 may be configured for use on a patient 513 lying on a patient table 512 to be treated in a minimally invasive manner. Robotic surgical system 500 may further include a database 514, in particular coupled to control device 504, in which are stored, for example, pre-operative data from patient 513 and/or anatomical atlases.

Each of the robot arms 502, 503 may include a plurality of members, which are connected through joints, and a mounted device which may be, for example, a surgical tool “ST.” One or more of the surgical tools “ST” may be instrument 10 (FIG. 1), thus providing such functionality on a robotic surgical system 500.

Robot arms 502, 503 may be driven by electric drives, e.g., motors, connected to control device 504. Control device 504, e.g., a computer, may be configured to activate the motors, in particular by means of a computer program, in such a way that robot arms 502, 503, and, thus, their mounted surgical tools “ST” execute a desired movement and/or function according to a corresponding input from manual input devices 507, 508, respectively. Control device 504 may also be configured in such a way that it regulates the movement of robot arms 502, 503 and/or of the motors.

Turning to FIGS. 4A-7B, as noted above, end effector assembly 40 includes first and second jaw members 42, 44, respectively, supported by the clevis portion of distal segment 32 of shaft 30. Each jaw member 42, 44, as also noted above, includes a proximal extension portion 43a, 45a and a distal body portion 43b, 45b, respectively, and is formed from a structural jaw 81a, 83a, an internal spacer (not shown), an outer housings 81b, 83b, and an electrically-conductive plate 81c, 83c defining the respective tissue-contacting surface 46, 48. Structural jaws 81a, 83a provides structural support to jaw members 42, 44 and include distal portions that support the components of distal body portions 43b, 45b of jaw members 42, 44, respectively, thereon, and proximal portions that extend proximally from distal body portions 43b, 45b to form proximal extension portion 43a, 45a of jaw members 42, 44. The distal portions of structural jaws 81a, 83a, more specifically, form distal body portions 43b, 45b of jaw members 42, 44 together with the internal spacers (not shown), outer housings 81b, 83b, and electrically-conductive plates 81c, 83c.

The proximal extension portion 43a, 45a of one of the jaw members, e.g., jaw member 44, may include a pair of spaced-apart flags 86a, 86b, while the proximal extension portion 43a, 45a of the other jaw member, e.g., jaw member 42, includes a single flag 84 received between the flags 86a, 86b of jaw member 44. Other configurations, e.g., the reverse configuration or configurations wherein both of proximal extension portions 43a, 45a include one or two flags, are also contemplated.

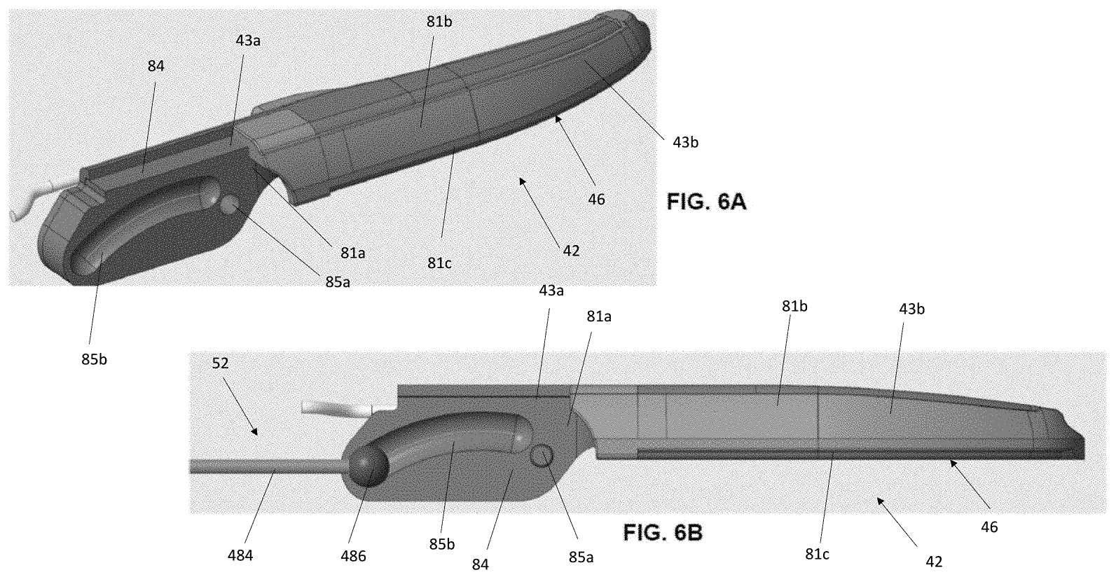

Referring to FIGS. 6A and 6B, flag 84 of proximal extension portion 43a of jaw member 42 defines a pivot aperture 85a and a cam groove 85b. Pivot aperture 85a may extend transversely through flag 84 or may be bifurcated to include disconnected pivot aperture portions on either side of flag 84. Alternatively, flag 84 may include pivot bosses protruding therefrom. Cam groove 85b may be curved, angled, combinations thereof, or otherwise configured along its length. Cam groove 85b defines a generally hemispherical transverse cross-sectional configuration such that the largest transverse dimension of cam groove 85b is defined at the open side thereof and such that the smallest transverse dimension of cam groove 85b is defined at the closed side thereof. Cam groove 85b is defined within a side surface of flag 84 such that the open side of cam groove 85b faces an interior surface of one of the flags 86a, 86b of proximal extension portion 45a of jaw member 44. Although illustrated as closed, it is also contemplated that the bottom of cam groove 85b can include a slot of smaller dimension than a dimension at the top of cam groove 85b.

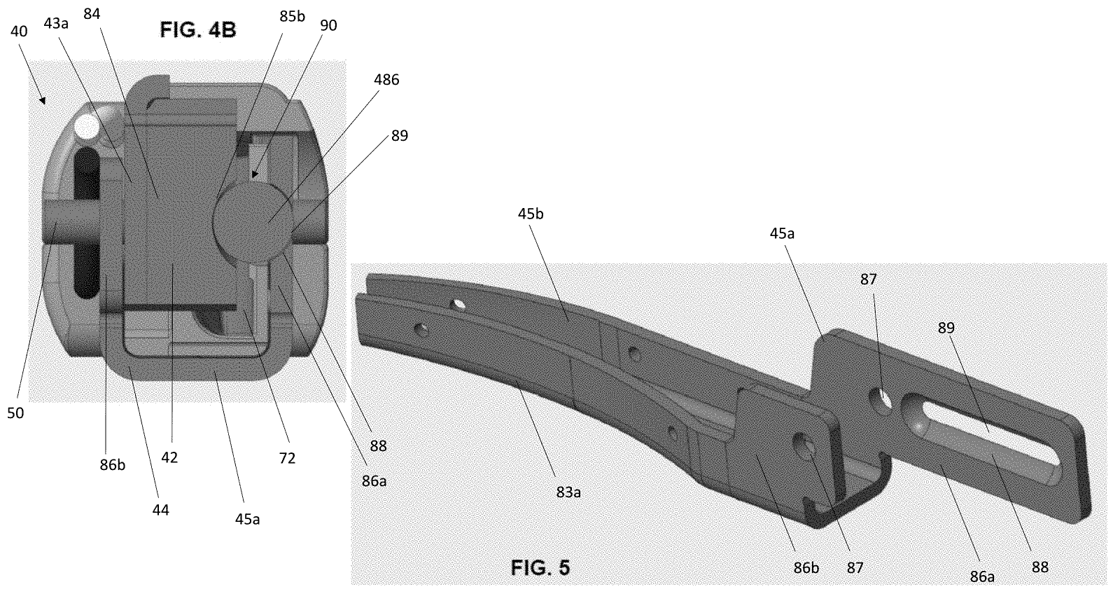

Referring to FIGS. 4A-5, flags 86a, 86b of proximal extension portion 45a of jaw member 44 each define pivot apertures 87 that are configured to align with pivot aperture 85a of flag 84 of proximal extension portion 43a of jaw member 42 to receive a pivot pin 50 therethrough to pivotably couple jaw members 42, 44 with one another, although jaw members 42, 44 may alternatively be pivotably coupled in any other suitable manner, e.g., via split pivot pin portions, pivot bosses extending from one of the jaw members 42, 44, or in any other suitable manner. Either or both of flags 86a, 86b of proximal extension portion 45a of jaw member 44 are secured, e.g., welded, or monolithically formed with the clevis portion of distal segment 32 of shaft 30 to thereby fix jaw member 44 relative to distal segment 32. Alternatively, in bilateral configurations, jaw member 44 may be coupled to shaft 30 in a pivotable manner, e.g., via pivot pin 50.

One of the flags 86a, 86b of proximal extension portion 45a, e.g., flag 86a, defines an elongated configuration relative to the other flag, e.g., flag 86b, such that flag 86a extends proximally beyond flag 86b. This elongated flag 86a defines a cam groove 88 which may be curved, angled, combinations thereof, or otherwise configured along its length. Cam groove 88 defines a generally hemispherical transverse cross-sectional configuration such that the largest transverse dimension of cam groove 88 is defined at the open side thereof and such that the smallest transverse dimension of cam groove 88 is defined at the substantially closed side thereof. Cam groove 88 is substantially closed in that it defines a slot 89 through the smallest transverse dimension side thereof so as to enable a reduced thickness of flag 86a without compromising the effective diameter of cam groove 88. However, it is also contemplated that cam groove 88 be fully closed at the smallest transverse dimension side thereof. Cam groove 88 is defined within an inwardly-facing side surface of flag 86a such that the open side of cam groove 88 faces the side surface of flag 84 that defines cam groove 85b with cam grooves 88, 85b at least partially overlapping one another. The overlapping portions of cam grooves 88, 85b cooperate to define a generally spherical cavity 90. In aspects, cam grooves 88, 85b define similar diameters; in other aspects, cam grooves 88, 85b define different diameters.

Referring again to FIGS. 4A-7B, as noted above, flag 84 of jaw member 42 is received between the flags 86a, 86b of jaw member 44. More specifically, flag 84 is configured, e.g., to define a suitable thickness, such that flag 84 alone and/or together with any other components disposed between flags 86a, 86b (or between elongated flag 86a and an internally-facing wall of the clevis portion of distal segment 32 of shaft 30) substantially fills the gap defined between flags 86a, 86b (or between elongated flag 86a and the internally-facing wall of the clevis portion of distal segment 32 of shaft 30). This configuration substantially inhibits lateral play of flag 84 and, thus, splay of jaw member 42. and also maintains the overlapping portions of cam grooves 88, 85b in close approximation relative to one another such that generally spherical cavity 90 is defined without a significant gap therebetween.

Cam drive mechanism 52, as noted above, enables pivoting of jaw member 42 relative to jaw member 44 and distal segment 32 of shaft 30 between the spaced-apart position (FIG. 7A) and the approximated position (FIG. 7B) for grasping tissue between tissue-contacting surfaces 46, 48. Cam drive mechanism 52 includes drive rod 484 and a drive ball 486. Drive rod 484 is coupled, directly or indirectly, to jaw drive assembly 400 of actuation assembly 100 (FIG. 2) such that drive rod 484 is translated proximally or distally based upon the rotational input provided to fourth input 140. Drive ball 486 is disposed at a distal end portion of drive rod 484 and is attached thereto via monolithic formation, welding, mechanical securement, or in any other suitable manner. Drive ball 486 defines a generally spherical shape and is configured for positioning within generally spherical cavity 90 defined by the overlapping portions of cam grooves 88, 85b. Drive ball 486 may define a diameter that generally approximates the diameter of generally spherical cavity 90, cam groove 88, and/or cam groove 85b and/or that is smaller or larger than the diameter of generally spherical cavity 90, cam groove 88, and/or cam groove 85b. Drive ball 486 defines a diameter larger than a height of slot 89 to inhibit passage of drive ball 486 therethrough. Regardless of the relative diameters, because the gap defined between flags 86a, 86b (or between elongated flag 86a and the internally-facing wall of the clevis portion of distal segment 32 of shaft 30), is substantially filled as detailed above, drive ball 486 is captured within generally spherical cavity 90 defined by the overlapping portions of cam grooves 88, 85b and is inhibited from escaping therefrom.

With drive ball 486 captured within generally spherical cavity 90 defined by the overlapping portions of cam grooves 85b, 88 of proximal extension portion 43a, 45a of jaw members 42, 44, respectively, and due to the orientation of cam grooves 85b, 88 relative to one another, translation of drive ball 486 relative to proximal extension portions 43a, 45a of jaw members 42, 44 urges drive ball 486 along cam grooves 85b, 88 to thereby pivot distal body portion 43b of jaw member 42 about pivot pin 50 and relative to distal body portion 45b of jaw member 44. More specifically, as shown in FIGS. 7A and 7B, proximal translation of drive ball 486, e.g., in response to proximal translation of drive rod 484 pulling drive ball 486 proximally, pivots distal jaw body portion 43b of jaw member 42 about pivot pin 52 and towards distal body portion 45b of jaw member 44, e.g., towards the approximated position (FIG. 7B), while distal translation of drive ball 486, e.g., in response to distal translation of drive rod 484 pushing drive ball 486 distally, pivots distal jaw body portion 43b of jaw member 42 about pivot pin 50 and away from distal body portion 45b of jaw member 44, e.g., towards the spaced-apart position (FIG. 7A).

The spherical configuration of drive ball 486 together with the hemispherical configurations of cam grooves 85b, 88 (and the spherical cavity 90 defined thereby), facilitate smooth translation of drive ball 486 cam grooves 85b, 88 and, thus, smooth pivoting of jaw member 42 relative to jaw member 44, while inhibiting binding throughout the entire jaw range of motion.

It will be understood that various modifications may be made to the aspects and features disclosed herein. Therefore, the above description should not be construed as limiting, but merely as exemplifications of various aspects and features. Those skilled in the art will envision other modifications within the scope and spirit of the claims appended thereto.

What is claimed is:

1. A surgical instrument, comprising:
a first jaw member defining a proximal portion including at least a first flag and a distal portion defining a tissue-contacting surface, the first flag defining a first cam groove having a hemispherical cross-sectional configuration;
a second jaw member defining a proximal portion including at least a second flag and a distal portion defining a tissue-contacting surface, the second flag defining a second cam groove having a hemispherical cross-sectional configuration, wherein the first and second jaw members are pivotably coupled to one another; and
a cam drive mechanism including a drive rod and a drive ball disposed at a distal end portion of the drive rod, the drive ball captured within the first and second cam grooves between the first and second flags,
wherein translation of the drive rod in a first direction slides the cam ball through the cam grooves to pivot the distal portion of at least one of the first or second jaw members relative to the distal portion of the other of the first or second jaw members towards an approximated position for grasping tissue between the tissue-contacting surfaces of the distal portions of the first and second jaw members.

2. The surgical instrument according to claim 1, further comprising a shaft having a distal segment, wherein the proximal portion of the second jaw member is fixed to the distal segment of the shaft.

3. The surgical instrument according to claim 2, wherein the shaft further comprises an articulating section proximal to the distal segment.

4. The surgical instrument according to claim 1, wherein the proximal portion of the second jaw member further includes a third flag spaced-apart from the second flag, wherein the first flag is disposed between the second and third flags.

5. The surgical instrument according to claim 4, wherein the first flag substantially occupies a space defined between the second and third flags to maintain the drive ball captured within the first and second cam grooves between the first and second flags.

6. The surgical instrument according to claim 1, wherein the first and second cam grooves at least partially intersect to form a spherical cavity for receipt of the drive ball therein.

7. The surgical instrument according to claim 1, wherein at least one of the tissue-contacting surfaces is adapted to connect to a source of electrosurgical energy for treating tissue grasped between the tissue-contacting surfaces.

8. A surgical instrument, comprising:
a housing;
a shaft extending distally from the housing;
an end effector assembly disposed at a distal end of the shaft, the end effector assembly including:
a first jaw member defining a proximal portion including at least a first flag and a distal portion defining a tissue-contacting surface, the first flag defining a first cam groove having a hemispherical cross-sectional configuration; and
a second jaw member defining a proximal portion including at least a second flag and a distal portion defining a tissue-contacting surface, the second flag defining a second cam groove having a hemispherical cross-sectional configuration, wherein the first and second jaw members are pivotably coupled to one another; and
a cam drive mechanism including a drive rod extending through the shaft and a drive ball disposed at a distal end portion of the drive rod, the drive ball captured within the first and second cam grooves between the first and second flags,
wherein translation of the drive rod in a first direction slides the cam ball through the cam grooves to pivot the distal portion of at least one of the first or second jaw members relative to the distal portion of the other of the first or second jaw members towards an approximated position for grasping tissue between the tissue-contacting surfaces of the distal portions of the first and second jaw members.

9. The surgical instrument according to claim 8, wherein the proximal portion of the second jaw member is fixed to a distal segment of the shaft.

10. The surgical instrument according to claim 9, wherein the shaft further comprises an articulating section proximal to the distal segment.

11. The surgical instrument according to claim 8, wherein the proximal portion of the second jaw member further includes a third flag spaced-apart from the second flag, wherein the first flag is disposed between the second and third flags.

12. The surgical instrument according to claim 11, wherein the first flag substantially occupies a space defined between the second and third flags to maintain the drive ball captured within the first and second cam grooves between the first and second flags.

13. The surgical instrument according to claim 8, wherein the first and second cam grooves at least partially intersect to form a spherical cavity for receipt of the drive ball therein.

14. The surgical instrument according to claim 8, wherein at least one of the tissue-contacting surfaces is adapted to connect to a source of electrosurgical energy for treating tissue grasped between the tissue-contacting surfaces.

15. The surgical instrument according to claim 8, wherein the housing includes a jaw drive mechanism disposed therein, the jaw drive mechanism operably coupled to the drive rod.

16. The surgical instrument according to claim 15, wherein the housing is configured to mount on a surgical robot, the surgical robot configured to operate the jaw drive mechanism to translate the drive rod.
